(19) United States
(12) Patent Application Publication
(10) Pub. No.: US 2007/0016184 A1
(43) Pub. Date: Jan. 18, 2007

(54) Medical-treatment electrode assembly and method for medical treatment

(75) Inventors: Michael S. Cropper, Edgewood, KY (US); Patrick A. Weizman, Liberty Township, OH (US); Paul T. Franer, Cincinnati, OH (US); David C. Yates, West Chester, OH (US); David K. Norvell, Monroe, OH (US); Thomas W. Huitema, Cincinnati, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: Ethicon Endo-Surgery, Inc.

(21) Appl. No.: 11/181,251

(22) Filed: Jul. 14, 2005

(51) Int. Cl. A61B 18/14

(52) U.S. Cl. 606/41

(57) ABSTRACT

One medical-treatment electrode assembly includes a medical-treatment flexible electrode supported on the outer surface of a sidewall of a flexible tube which is insertable into a patient. Another assembly includes two electrodes supported on the outer surface of a flexible tube, wherein a video camera of a flexible endoscope inserted into the tube can view patient tissue between the two electrodes. An additional assembly includes apparatus for moving patient tissue into a sidewall opening of a tube which supports a medical-treatment electrode to tighten patient tissue outside the tube against the medical-treatment electrode. A method for medical treatment includes moving patient tissue into a sidewall opening of a tube which supports a medical-treatment electrode to tighten patient tissue outside the tube into substantially full contact with the medical-treatment electrode.

### CROSS REFERENCE TO RELATED PATENT APPLICATION

This patent application incorporates by reference: US Patent Application Publication 2002/0177847 published Nov. 28, 2002; US Patent Application Publication 2003/0181900 published Sep. 25, 2003; US Patent Application Publication 2003/0181905 published Sep. 25, 2003; US Patent Application Publication 2003/0216727 published Nov. 20, 2003; U.S. patent application Ser. No. 10/105,722 filed Mar. 25, 2002; U.S. patent application Ser. No. 10/887,646 filed Jul. 9, 2004; U.S. patent application Ser. No. 10/934,674 filed Sep. 3, 2004; and U.S. patent application Ser. No. 10/939,726 filed Sep. 13, 2004.

### FIELD OF THE INVENTION

The present invention is related generally to medical systems, and more particularly to a medical-treatment electrode assembly and to a method for medical treatment.

### BACKGROUND OF THE INVENTION

A medical-treatment electrode assembly is known wherein a tube having two electrodes is inserted into a patient's esophagus, wherein the two electrodes are operatively connected to a medical radio-frequency (RF) generator, and wherein the two electrodes are brought into contact with esophageal tissue to treat gastro-esophageal reflux disease and other diseases of mucosal tissue.

Still, scientists and engineers continue to seek improved medical-treatment electrode assemblies and methods for medical treatment.

### SUMMARY

A first expression of a first embodiment of a medical-treatment electrode assembly of the invention includes a flexible tube and a first medical-treatment flexible electrode. The flexible tube includes a sidewall having an outer surface and includes a distal end insertable into a patient. The first medical-treatment flexible electrode is fixedly supported on the outer surface of the sidewall of the flexible tube proximate the distal end of the flexible tube. The first medical-treatment flexible electrode is contactable with patient tissue. The first medical-treatment flexible electrode is operatively connectable to a medical radio-frequency (RF) generator.

A second expression of a first embodiment of a medical-treatment electrode assembly of the invention includes a flexible tube and two medical-treatment electrodes. The flexible tube has an outer surface and has a distal end insertable into a patient. The two medical-treatment electrodes are supported on the outer surface of the flexible tube proximate the distal end, are contactable with patient tissue, and are operatively connectable to a medical radio-frequency (RF) generator, wherein the two medical-treatment electrodes are spaced apart, and wherein a video camera of a flexible endoscope inserted into the flexible tube proximate the distal end can view the patient tissue between the two medical-treatment electrodes.

A first expression of a second embodiment of a medical-treatment electrode assembly of the invention includes a first medical-treatment electrode body. The first medical-treatment electrode body is insertable into a patient and is operatively connectable to a medical radio-frequency (RF) generator. The first medical-treatment electrode body has an outer surface contactable with patient tissue, has a central lumen operatively connectable to a vacuum source, and has an opening extending from the outer surface to the central lumen.

An expression of a third embodiment of a medical-treatment electrode assembly of the invention includes a tube, a medical-treatment electrode, and patient-tissue-moving apparatus. The tube is insertable into a patient and includes a sidewall having an opening. The medical-treatment electrode is supported by the tube, is contactable with patient tissue which is outside the tube, and is operatively connectable to a medical radio-frequency (RF) generator. The patient-tissue-moving apparatus moves patient tissue into the opening to tighten patient tissue outside the tube against the medical-treatment electrode.

A method of the invention is for medical treatment and includes inserting a tube into a hollow body organ of a patient, wherein the tube supports a medical-treatment electrode and has a sidewall opening. The method also includes then moving patient tissue into the sidewall opening to tighten patient tissue outside the tube into substantially full contact with the medical-treatment electrode. The method also includes then activating the medical-treatment electrode.

Several benefits and advantages are obtained from one or more of the expressions of one or more of the embodiments and method of the invention. In one application, having a medical-treatment electrode which is flexible provides more intimate contact between the electrode and patient tissue which reduces charring of patient tissue and which improves non-visual monitoring of tissue treatment. In the same or a different application, being able to have a video camera of a flexible endoscope view patient tissue between two medical-treatment electrodes allows the user to visually monitor tissue treatment for patient tissue between the two medical-treatment electrodes. In one implementation, having a medical-treatment electrode body with a central lumen operatively connectable to a vacuum source and with an opening extending from the outer surface of the medical-treatment electrode body to the central lumen provides a vacuum to draw patient tissue into more intimate contact with the electrode. In one employment, having patient-tissue-apparatus apparatus for moving patient tissue into a sidewall opening of a tube supporting a medical-treatment electrode tightens patient tissue outside the tube against, and into substantially full contact with, the medical-treatment electrode.

### BRIEF DESCRIPTION OF THE FIGURES

FIG. 1 is a schematic, longitudinal cutaway view of an embodiment of a medical instrument which includes a first embodiment of a medical-treatment electrode assembly of the invention and which includes a medical radio-frequency (RF) generator and an embodiment of a flexible endoscope;

FIG. 2 is a view of a portion of the medical-treatment electrode assembly of FIG. 1, taken along lines 2-2 in FIG. 1;

FIG. 3 is a schematic, top planar view of a second embodiment of a medical-treatment electrode assembly of the invention;

FIG. 4 is a cross-sectional view of the medical-treatment electrode assembly of FIG. 3 taken along lines 4-4 in FIG. 3;

FIGS. 5-7 are schematic, radial cross-sectional views of a patient's esophagus and a third embodiment of a medical-treatment electrode assembly of the invention showing various stages of patient esophageal tissue being drawn into a tube opening and of patient esophageal tissue outside the tube being tightened against two medical-treatment electrodes;

FIG. 8 is a schematic, longitudinal cross-sectional view of a portion of a patient's esophagus and a cutaway view of a fourth embodiment of a medical-treatment electrode assembly of the invention showing patient tissue being drawn in a tube opening by grasping forceps and showing patient tissue outside the tube being tightened against a medical-treatment electrode supported by the tube;

FIG. 9 is a side elevational view of a corkscrew retractor; and

FIG. 10 is a side elevational view of a hook retractor.

### DETAILED DESCRIPTION

Before explaining several embodiments of the present invention in detail, it should be noted that each embodiment is not limited in its application or use to the details of construction and arrangement of parts and steps illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

It is further understood that any one or more of the following-described embodiments, expressions of embodiments, examples, etc. can be combined with any one or more of the other following-described embodiments, expressions of embodiments, examples, etc.

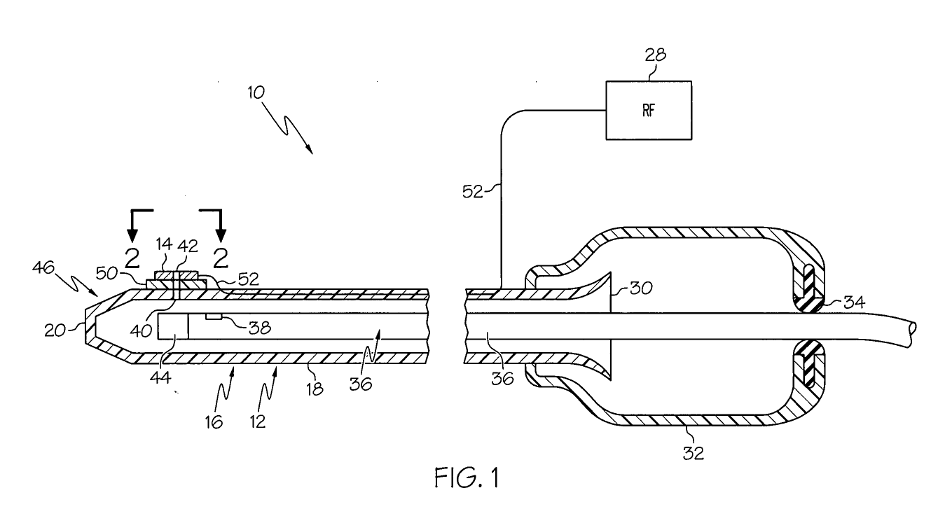

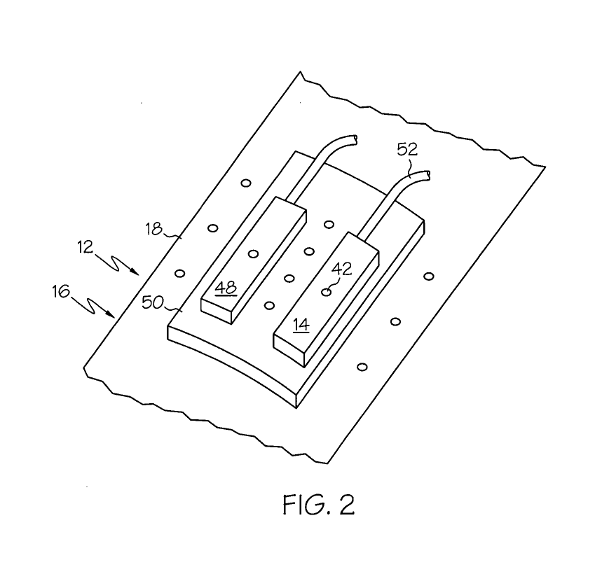

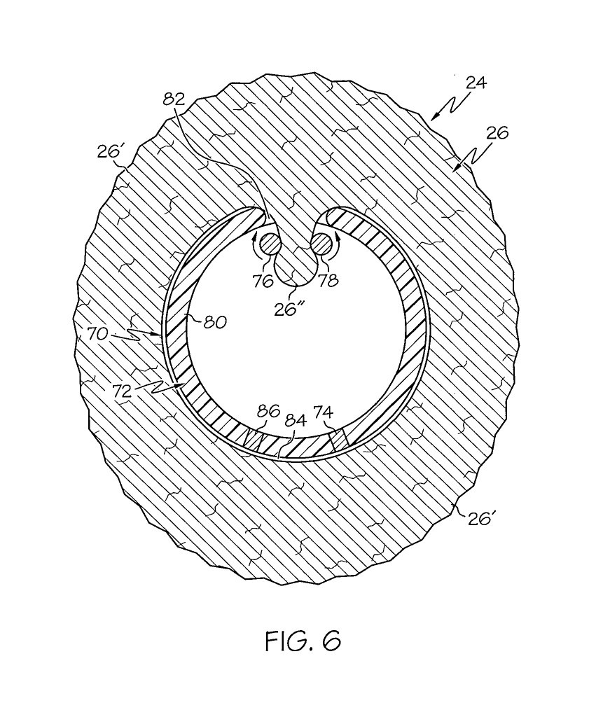

A first embodiment of a medical-treatment electrode assembly 10 of the invention is shown in FIGS. 1 and 2. A first expression of the embodiment of FIGS. 1 and 2 is for a medical-treatment electrode assembly 10 including a flexible tube 12 and a first (i.e., at least one) medical-treatment flexible electrode 14. The flexible tube 12 includes a sidewall 16 having an outer surface 18 and includes a distal end 20 insertable into a patient (such as, without limitation, insertable into the esophagus 22 of a patient 24 shown in FIG. 5 for a third embodiment to be described later). The first medical-treatment flexible electrode 14 is fixedly supported (directly or indirectly) on the outer surface 18 proximate the distal end 20, is contactable with patient tissue (such as patient tissue 26 shown in FIG. 5), and operatively connectable to a medical radio-frequency (RF) generator 28.

In one application, having a medical-treatment electrode which is flexible provides more intimate contact between the electrode and patient tissue which reduces charring of patient tissue and which improves non-visual monitoring of tissue treatment.

In one enablement of the first expression of the embodiment of FIGS. 1 and 2, the flexible tube 12 has a proximal end 30 disposable outside the patient 24. In this enablement, the medical-treatment electrode assembly 10 also includes a handle 32 surrounding, and attached to, the flexible tube 12 proximate the proximal end 30 and includes an annular seal 34 attached to the handle 32. The annular seal 34 is adapted to sealing receive a flexible endoscope 36 insertable into the flexible tube 12.

In one arrangement, wherein a flexible endoscope 36 is employed, the flexible endoscope 36 includes an aspiration port 38, and the sidewall 16 includes a through hole 40 disposed proximate the distal end 20 and in fluid communication with the aspiration port 38 of the flexible endoscope 36. In one variation, the first medical-treatment flexible electrode 14 includes a through hole 42 aligned with the through hole 38 of the sidewall 16. In one application, providing a vacuum draws patient tissue into more intimate contact with the electrode.

In the same or a different arrangement, wherein a flexible endoscope 36 is employed, the flexible endoscope 36 includes a video camera 44, and the flexible tube 12 includes a distal end cap (e.g., 46) attached to the distal end of the sidewall 16. In one construction, the sidewall 16 is a monolithic sidewall extending from the distal end cap to the proximal end 30 of the flexible tube 12. In one variation, the distal end cap and the sidewall 16 are portions of a monolithic flexible tube 12. In one example, the distal end cap is chosen from the group consisting of a flexible tapered closed end cap 46, an open end cap (not shown) adapted to allow passage therethrough of the video camera of the flexible endoscope, and an end cap (not shown) adapted to open to allow passage therethrough of the video camera of the flexible endoscope and to close upon removal of the flexible endoscope therefrom.

In one employment of the first expression of the embodiment of FIGS. 1 and 2, a lead 52 operatively connects the first medical-treatment flexible electrode 14 to the radio-frequency (RF) generator 28. In one illustration, the lead 52 extends through a longitudinal channel in the sidewall 16 of the flexible tube 12. In one variation, the lead 52 from the electrode exits the flexible tube 12 through the sidewall 16 before reaching the handle 32. In another variation, not shown, the lead from the electrode exits the flexible tube 12 inside the handle 32 and then exits the handle 32. In one modification, not shown, the lead is operatively connected to a control button on the handle to start and stop the medical treatment. In one method, the lead and substrate are manufactured using etched circuit technology, wherein the lead is substantially flat and comprises, consists essentially of, or consists of copper, and the substrate is substantially flat and comprises, consists essentially of, or consists of polyester. In one deployment, shrink wrap, not shown, surrounds the longitudinal juncture of the flexible tube and the distal end of the handle.

In one extension of the first expression of the embodiment of FIGS. 1 and 2, the medical-treatment electrode assembly 10 also includes a second medical-treatment flexible electrode 48. The second medical-treatment flexible electrode 48 is supported (directly or indirectly) on the outer surface 18 proximate the distal end 20 and is spaced apart from the first medical-treatment flexible electrode 14. The second medical-treatment flexible electrode 48 is contactable with the patient tissue 26 and is operatively connectable to the medical radio-frequency (RF) generator 28. It is noted that when only a single electrode is present, the assembly is operated as a monopolar assembly, and when two (or more) electrodes are present, the assembly can be operated as a monopolar and/or a bipolar assembly as can be appreciated by the artisan.

A second expression of the embodiment of FIGS. 1 and 2 is for a medical-treatment electrode assembly 10 including a flexible tube 12 and two medical-treatment (flexible or rigid) electrodes 14 and 48. The flexible tube 12 has an outer surface 18 and has a distal end 20 insertable into a patient 24. The two medical-treatment electrodes 14 and 48 are supported (directly or indirectly) on the outer surface 18 proximate the distal end 20, are contactable with patient tissue 26, and are operatively connectable to a medical radio-frequency (RF) generator 28. The two medical-treatment electrodes 14 and 48 are spaced apart, and a video camera 44 of a flexible endoscope 36 inserted into the flexible tube 12 and translated proximate the distal end 20 can view the patient tissue 26 between the two medical-treatment electrodes 14 and 48.

In one application, being able to have a video camera of a flexible endoscope view patient tissue between two medical-treatment electrodes allows the user to visually monitor tissue treatment for patient tissue between the two medical-treatment electrodes.

It is noted that the enablements, arrangements, variations, etc. of the previously-described first expression of the embodiment of FIGS. 1 and 2 are equally applicable to the second expression of the embodiment of FIGS. 1 and 2.

In one construction of the second expression of the embodiment of FIGS. 1 and 2, the flexible tube 12 is chosen from the group consisting of a transparent tube (as shown), a tube (not shown) having a solid transparent window disposed between the two medical-treatment electrodes, and a tube (not shown) having a tube cutout disposed between the two medical-treatment electrodes. In one example, when the flexible tube 12 is a transparent tube, the flexible tube 12 comprises, consists essentially of, or consists of polyethylene, polyurethane, or polyester. In one variation, the medical-treatment electrode assembly 10 also includes a transparent substrate 50 bonded to the outer surface 18 of the flexible tube 12, wherein the two medical-treatment electrodes 14 and 48 are bonded to the substrate 50. In one modification, the lead 52 is bonded to the substrate 50, and the substrate 50 (with the bonded lead 52) extends (not shown) on the outer surface 18 to proximate the proximal end 30 of the flexible tube 12. In one example, the substrate 50 comprises, consists essentially of, or consists of polyester. It is noted that additional unmarked through holes (similar to through holes 40 and 42) are shown as small circles on the second medical-treatment electrode 48, on the substrate 50, and on the flexible tube 12 in FIG. 2. The number and layout of the through holes are left to the artisan.

A third expression of the embodiment of FIGS. 1 and 2 is for a medical-treatment electrode assembly including a flexible tube 12 and two medical-treatment flexible electrodes 14 and 48. The flexible tube 12 has an outer surface 18 and has a distal end 20 insertable into a patient 24. The two medical-treatment flexible electrodes 14 and 48 are fixedly supported on the outer surface 18 proximate the distal end 20, are contactable with patient tissue 26, and are operatively connectable to a medical radio-frequency (RF) generator 28. The two medical-treatment flexible electrodes 14 and 48 are spaced apart. A video camera 44 of a flexible endoscope 36 inserted into the flexible tube 12 and translated proximate the distal end 20 can view the patient tissue 26 between the two medical-treatment flexible electrodes 14 and 48.

It is noted that the enablements, arrangements, variations, constructions, etc. of the previously-described first and/or second expressions of the embodiment of FIGS. 1 and 2 are equally applicable to the third expression of the embodiment of FIGS. 1 and 2.

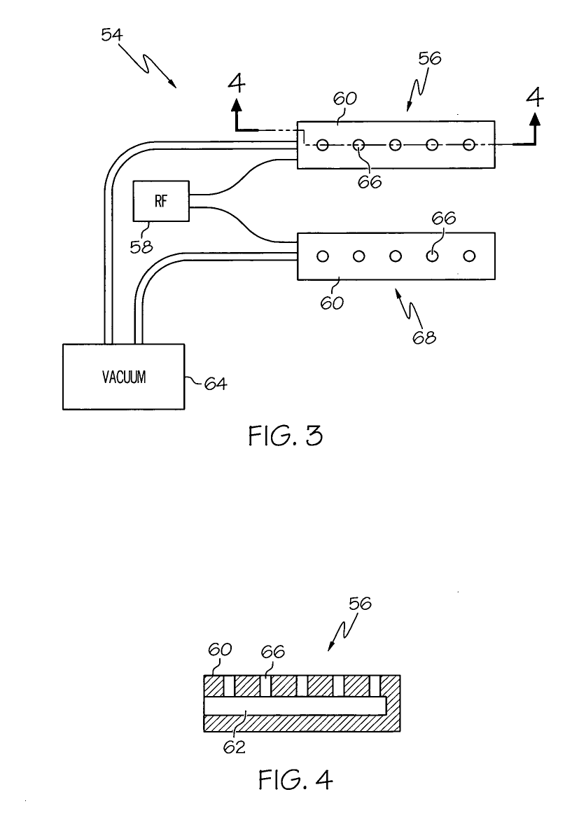

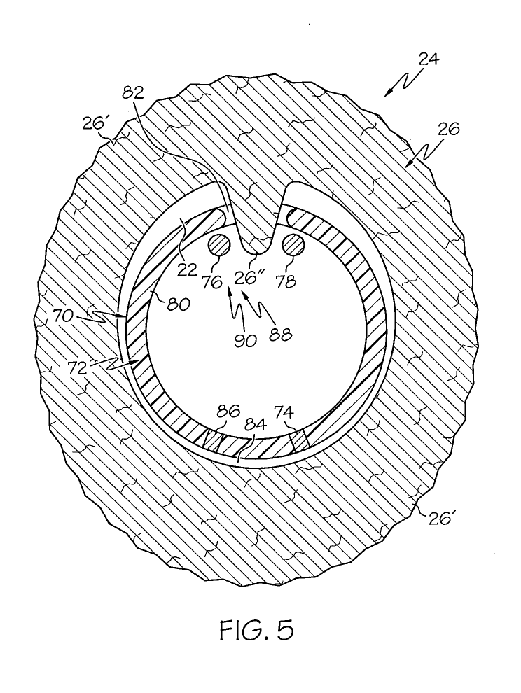

A second embodiment of a medical-treatment electrode assembly 54 of the invention is shown in FIGS. 3 and 4. A first expression of the embodiment of FIGS. 3 and 4 is for a medical-treatment electrode assembly 54 including a first medical-treatment electrode body 56 which is insertable into a patient 24. The first medical-treatment electrode body 56 is operatively connectable to a medical radio-frequency (RF) generator 58 and has an outer surface 60 contactable with patient tissue 26. The first medical-treatment electrode body 56 has a central lumen 62 operatively connectable to a vacuum source 64 and has an opening 66 extending from the outer surface 60 to the central lumen 62.

In one application, providing a vacuum draws patient tissue into more intimate contact with the electrode. Examples of vacuum sources 64 include, without limitation, syringes, squeeze bulbs, and pump motors.

In one extension of the first expression of the embodiment of FIGS. 3 and 4, the medical-treatment electrode assembly 54 also includes a second medical-treatment electrode body 68 insertable into the patient 24 and spaced apart from the first medical-treatment electrode body 56. The second medical-treatment electrode body 68 is operatively connectable to the medical radio-frequency (RF) generator 58 and has an outer surface 60 contactable with patient tissue 26. The second medical-treatment electrode body 60 has a central lumen (similar to the central lumen 62 of the first medical-treatment electrode body 56) operatively connectable to the vacuum source 64 and has an opening 66 extending from the outer surface 60 of the second medical-treatment electrode body 68 to the central lumen of the second medical-treatment electrode body 68.

In one application, the first and second medical-treatment electrode bodies 56 and 68 are supported on the outside of a flexible tube (not shown) in a manner similar to that shown for the embodiment of FIGS. 1 and 2.

A third embodiment of a medical-treatment electrode assembly 70 of the invention is shown in FIGS. 5-7. A first expression of the embodiment of FIGS. 5-7 is for a medical-treatment electrode assembly 70 including a flexible tube 72 (shown in cross-section), a medical-treatment electrode 74, and two rollers 76 and 78. The flexible tube 72 is insertable into a patient 24 and includes a sidewall 80 having an opening 82. The medical-treatment electrode 74 is supported by the flexible tube 72 and is contactable with patient tissue 26′ which is outside the flexible tube 72. The medical-treatment electrode 74 is operatively connectable to a medical radio-frequency (RF) generator (such as RF generator 58 of FIG. 3), and the flexible tube 72 is operatively connectable to a vacuum source (such as vacuum source 64 of FIG. 3) to draw patient tissue 26″ into the opening 82. The two rollers 76 and 78 are disposed inside the flexible tube 72 and are adapted to rollingly engage patient tissue 26″, drawn into the opening 82 by the vacuum source, to draw more patient tissue 26″ into the flexible tube 72 through the opening 82 to tighten patient tissue 26′ outside the flexible tube 72 against the medical-treatment electrode 74.

In one configuration of the first expression of the embodiment of FIGS. 5-7, the opening 82 is located closer to the two rollers 76 and 78 than to the medical-treatment electrode 74. In the same or a different configuration, the two rollers 76 and 78 are translatable toward and away from each other. Mechanisms for rotating and translating the two rollers 76 and 78 are left to the artisan.

In one procedure employing the first expression of the embodiment of FIGS. 5-7, the flexible tube 72 of the medical-treatment electrode assembly 70 is inserted into the esophagus 22 of a patient 24 to have a few cellular layers of patient tissue 26″ outside the flexible tube 72 be medically treated by the medical-treatment electrode 74. In one example, as shown in FIG. 5, at first some patient tissue 26″ is drawn through the opening 82 and into the flexible tube 72 by vacuum alone with the two rollers 76 and 78 spaced apart enough so as not to contact such patient tissue 26″. In this example, as shown in FIG. 6, then the two rollers 76 and 78 are brought toward each other to rollingly engage such patient tissue 26″ to begin to draw more patient tissue 26″ through the opening 82 and into the flexible tube 72. This action increases the tightening around the flexible tube 72 of patient tissue 26′ outside the flexible tube 72. In this example, as shown in FIG. 7, the two rollers 76 and 78 have drawn enough patient tissue 26″ through the opening 82 and into the flexible tube 72 to cause patient tissue 26′ outside the flexible tube 72 to more intimately contact the medical-treatment electrode 74. Notice how the gap 84 between the outside of the flexible tube 72 and the patient tissue 26′ outside the flexible tube 72 decreases from FIG. 5 to FIG. 6 to FIG. 7, and that the gap 84 disappears in FIG. 7 between the patient tissue 26′ outside the flexible tube 72 and the outside of the flexible tube 72 at the medical-treatment electrode 74.

It is noted that, in the example, the direction of rotation of the two rollers 76 and 78 to draw more patient tissue 26″ through the opening 82 and into the flexible tube 72 is shown by unmarked arrows in FIGS. 6 and 7. In one construction, the surface of the two rollers 76 and 78 is textured to better grip patient tissue 26″.

A second expression of the embodiment of FIGS. 5-7 is for a medical-treatment electrode assembly 70 including a flexible tube 72, two medical-treatment electrodes 74 and 86, and two rollers 76 and 78. The flexible tube 72 is insertable into a patient 24 and includes a sidewall 80 having an opening 82. The two medical-treatment electrodes 74 and 86 are supported by the flexible tube 72, are spaced apart, and are contactable with patient tissue 26′ which is outside the flexible tube 72. The two medical-treatment electrodes 74 and 86 are operatively connectable to a medical radio-frequency (RF) generator (such as RF generator 58 of FIG. 3), and the flexible tube 72 is operatively connectable to a vacuum source (such as vacuum source 64 of FIG. 3) to draw patient tissue 26″ into the opening 82. The two rollers 76 and 78 are disposed inside the flexible tube 72 and are adapted to rollingly engage patient tissue 26″, drawn into the opening 82 by the vacuum source, to draw more patient tissue 26″ into the flexible tube 72 through the opening 82 to tighten patient tissue 26′ outside the flexible tube 72 against the two medical-treatment electrodes 74 and 86.

In one configuration of the second expression of the embodiment of FIGS. 5-7, the opening 82 is located closer to the two rollers 76 and 78 than to the two medical-treatment electrodes 74 and 86. In the same or a different configuration, the two rollers 76 and 78 are translatable toward and away from each other.

In one deployment of the second expression of the embodiment of FIGS. 5-7, the two rollers 76 and 78 are adapted to rollingly engage esophageal patient tissue 26″, and wherein the two medical-treatment electrodes 74 and 86 are contactable with esophageal patient tissue 26′ which is outside the flexible tube 72.

A third expression of the embodiment of FIGS. 5-7 is for a medical-treatment electrode assembly 70 which includes a tube 72 and a medical-treatment electrode 74. The tube 72 is insertable into a patient 24 and includes a sidewall 80 having an opening 82. The medical-treatment electrode 74 is supported by the tube 72, is contactable with patient tissue 26′ which is outside the tube 72, and is operatively connectable to a medical radio-frequency (RF) generator (such as RF generator 28 of FIG. 1 or RF generator 58 of FIG. 3). The medical-treatment electrode assembly 70 also includes means 88 for moving patient tissue 26″ into the opening 82 to tighten patient tissue 26′ outside the tube 72 against the medical-treatment electrode 74.

In one example, the patient-tissue-moving means 88 includes a vacuum source (such as vacuum source 64 shown in FIG. 3). In the same or a different example, the patient-tissue-moving means 88 includes two rollers 76 and 78. In one variation, the means 88 includes only one of the vacuum source and the rollers. In a different variation, the means 88 includes both the vacuum source and the rollers. Other patient-tissue-moving means 88 includes a tissue grasper, a tissue pincher, a tissue pusher, tissue tweezers, and the like.

In one enablement, the patient-tissue-moving means 88 includes a tissue engaging device 90 (such as, but not limited to, rollers 76 and 78) adapted to mechanically pull and/or push patient tissue 26″ into the opening 82 to tighten patient tissue 26′ outside the tube 72 against the medical-treatment electrode 74. In one implementation, the tissue engaging device 90 is translatable and/or rotatable to mechanically pull and/or push patient tissue 26″ into the opening 82 to tighten patient tissue 26′ outside the tube 72 against the medical-treatment electrode 74. In one construction, the tissue engaging device 90 is disposed at the distal end of a flexible or rigid endoscope (not shown) which is insertable into the tube 72 of FIGS. 5-7 and which is operable from outside the patient.

Examples of tissue engaging devices 90 include, without limitation, the previously-described rollers 76 and 78, tissue gripping devices, tissue tweezing devices, tissue clamping devices, tissue squeezing devices, tissue reversibly penetrating devices, tissue holding devices, tissue pressing devices, and the like. Examples of tissue clamping devices include, without limitation, grasping forceps, forked jaw grasping forceps, rat tooth grasping forceps, three prong grasping forceps, tripod grasping forceps, fenestrated cup forceps, ellipsoid forceps, and the like. Examples of tissue reversibly penetrating devices include, without limitation, corkscrew retractors, hook retractors, and the like.

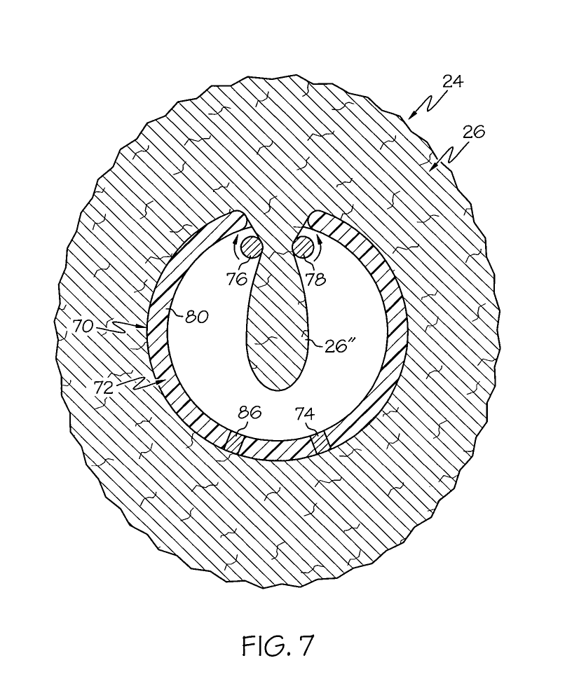

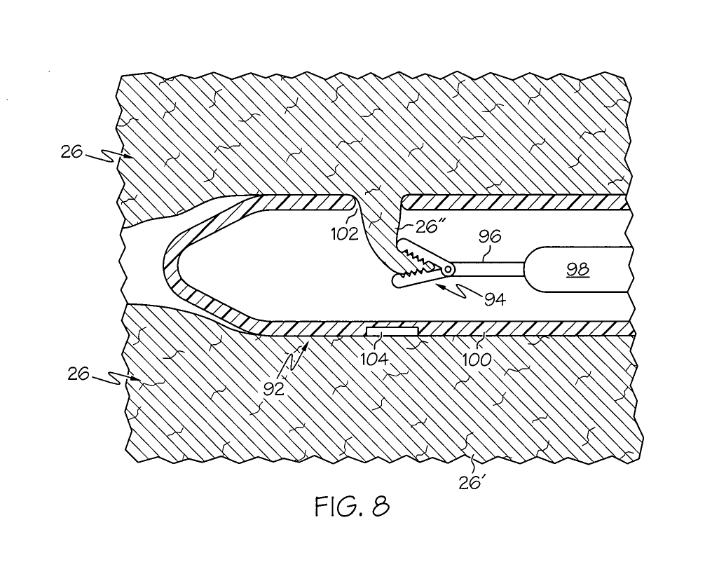

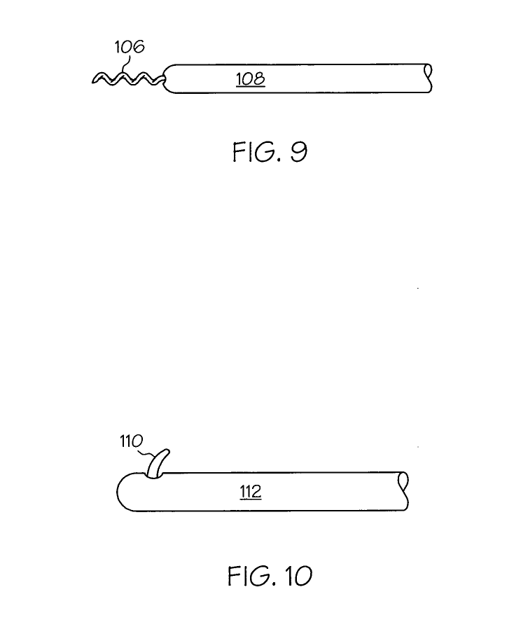

One embodiment of a medical-treatment electrode assembly 92 employing grasping forceps 94 is shown in FIG. 8. The grasping forceps 94 are attached to the end of a translatable shaft 96 extending from the distal end of a flexible endoscope 98 disposed in a tube 100 having an opening 102. The grasping forceps 94 are shown pulling patient tissue 26″ into the opening 102 to tighten patient tissue 26′ outside the tube 100 against the medical-treatment electrode 104 which is supported by the tube 100. An embodiment of a corkscrew retractor 106 extending from the distal end of an endoscope 108 is shown in FIG. 9, and an embodiment of a hook retractor 110 extending from a distal end portion of an endoscope 112 is shown in FIG. 10. In one variation, the corkscrew retractor 106 and the hook retractor 110 are made of a shape memory alloy for easier insertion within the endoscope 108/112.

A first method of the invention is for medical treatment and includes inserting a tube into a hollow body organ of a patient, wherein the tube supports a medical-treatment electrode and has a sidewall opening. The first method includes then moving patient tissue into the sidewall opening to tighten patient tissue outside the tube into substantially full contact with the medical-treatment electrode. The first method includes then activating the medical-treatment electrode.

A second method of the invention is for medical treatment and includes inserting a tube into an abdominal or thoracic cavity of a patient, wherein the tube supports a medical-treatment electrode and has a sidewall opening. The second method includes then moving patient tissue into the sidewall opening to tighten patient tissue outside the tube into substantially full contact with the medical-treatment electrode. The second method includes then activating the medical-treatment electrode.

Several benefits and advantages are obtained from one or more of the expressions of one or more of the embodiments and method of the invention. In one application, having a medical-treatment electrode which is flexible provides more intimate contact between the electrode and patient tissue which reduces charring of patient tissue and which improves non-visual monitoring of tissue treatment. In the same or a different application, being able to have a video camera of a flexible endoscope view patient tissue between two medical-treatment electrodes allows the user to visually monitor tissue treatment for patient tissue between the two medical-treatment electrodes. In one implementation, having a medical-treatment electrode body with a central lumen operatively connectable to a vacuum source and with an opening extending from the outer surface of the medical-treatment electrode body to the central lumen provides a vacuum to draw patient tissue into more intimate contact with the electrode. In one employment, having patient-tissue-moving apparatus for moving patient tissue into a sidewall opening of a tube supporting a medical-treatment electrode tightens patient tissue outside the tube against, and into substantially full contact with, the medical-treatment electrode.

While the present invention has been illustrated by a description of several embodiments and examples, etc. thereof, it is not the intention of the applicants to restrict or limit the spirit and scope of the appended claims to such detail. Numerous other variations, changes, and substitutions will occur to those skilled in the art without departing from the scope of the invention. It will be understood that the foregoing description is provided by way of example, and that other modifications may occur to those skilled in the art without departing from the scope and spirit of the appended Claims.

What is claimed is:

1. A medical-treatment electrode assembly comprising:
a) a flexible tube including a sidewall having an outer surface and including a distal end insertable into a patient; and
b) a first medical-treatment flexible electrode fixedly supported on the outer surface proximate the distal end, contactable with patient tissue, and operatively connectable to a medical radio-frequency (RF) generator.

2. The medical-treatment electrode assembly of claim 1, wherein the flexible tube has a proximal end disposable outside the patient, and also including a handle surrounding and attached to the flexible tube proximate the proximal end and including an annular seal attached to the handle, wherein the annular seal is adapted to sealing receive a flexible endoscope insertable into the flexible tube.

3. The medical-treatment electrode assembly of claim 2, wherein the flexible endoscope includes an aspiration port, and wherein the sidewall includes a through hole disposed proximate the distal end and in fluid communication with the aspiration port of the flexible endoscope.

4. The medical-treatment electrode assembly of claim 3, wherein the first medical-treatment flexible electrode includes a through hole aligned with the through hole of the sidewall.

5. The medical-treatment electrode assembly of claim 2, wherein the flexible tube includes a distal end cap, and wherein the distal end cap is chosen from the group consisting of a flexible tapered closed end cap, an open end cap adapted to allow passage therethrough of the video camera of the flexible endoscope, and an end cap adapted to open to allow passage therethrough of the video camera of the flexible endoscope and to close upon removal of the flexible endoscope therefrom.

6. The medical-treatment electrode assembly of claim 1, also including a second medical-treatment flexible electrode supported on the outer surface proximate the distal end, spaced apart from the first medical-treatment flexible electrode, contactable with the patient tissue, and operatively connectable to the medical radio-frequency (RF) generator.

7. A medical-treatment electrode assembly comprising:
a) a flexible tube having an outer surface and having a distal end insertable into a patient; and
b) two medical-treatment electrodes supported on the outer surface proximate the distal end, contactable with patient tissue, and operatively connectable to a medical radio-frequency (RF) generator, wherein the two medical-treatment electrodes are spaced apart, and wherein a video camera of a flexible endoscope inserted into the flexible tube and translated proximate the distal end can view the patient tissue between the two medical-treatment electrodes.

8. The medical-treatment electrode assembly of claim 7, wherein the flexible tube has a proximal end disposable outside the patient, and also including a handle surrounding and attached to the flexible tube proximate the proximal end and including an annular seal attached to the handle, wherein the annular seal is adapted to sealing receive a flexible endoscope insertable into the flexible tube.

9. The medical-treatment electrode assembly of claim 7, wherein the flexible endoscope includes an aspiration port, and wherein the flexible tube includes a through hole disposed proximate the distal end and in fluid communication with the aspiration port of the flexible endoscope.

10. The medical-treatment electrode assembly of claim 9, wherein at least one of the two medical-treatment flexible electrodes includes a through hole aligned with the through hole of the sidewall.

11. The medical-treatment electrode assembly of claim 7, wherein the flexible tube includes a distal end cap, and wherein the distal end cap is chosen from the group consisting of a flexible tapered closed end cap, an open end cap adapted to allow passage therethrough of the video camera of the flexible endoscope, and an end cap adapted to open to allow passage therethrough of the video camera of the flexible endoscope and to close upon removal of the flexible endoscope therefrom.

12. The medical-treatment electrode assembly of claim 7, wherein the flexible tube is chosen from the group consisting of a transparent tube, a tube having a solid transparent window disposed between the two medical-treatment electrodes, and a tube having a tube cutout disposed between the two medical-treatment electrodes.

13. The medical-treatment electrode assembly of claim 12, also including a transparent substrate bonded to the outer surface of the flexible tube, wherein the two medical-treatment electrodes are bonded to the substrate.

14. A medical-treatment electrode assembly comprising:
a) a flexible tube having an outer surface and having a distal end insertable into a patient; and
b) two medical-treatment flexible electrodes fixedly supported on the outer surface proximate the distal end, contactable with patient tissue, and operatively connectable to a medical radio-frequency (RF) generator, wherein the two medical-treatment flexible electrodes are spaced apart, and wherein a video camera of a flexible endoscope inserted into the flexible tube and translated proximate the distal end can view the patient tissue between the two medical-treatment flexible electrodes.

15. The medical-treatment electrode assembly of claim 14, wherein the flexible tube has a proximal end disposable outside the patient, and also including a handle surrounding and attached to the flexible tube proximate the proximal end and including an annular seal attached to the handle, wherein the annular seal is adapted to sealing receive a flexible endoscope insertable into the flexible tube.

16. The medical-treatment electrode assembly of claim 14, wherein the flexible endoscope includes an aspiration port, and wherein the flexible tube includes a through hole disposed proximate the distal end and in fluid communication with the aspiration port of the flexible endoscope.

17. The medical-treatment electrode assembly of claim 16, wherein a least one of the two medical-treatment flexible electrodes includes a through hole aligned with the through hole of the sidewall.

18. The medical-treatment electrode assembly of claim 14, wherein the flexible tube includes a distal end cap, and wherein the distal end cap is chosen from the group consisting of a flexible tapered closed end cap, an open end cap adapted to allow passage therethrough of the video camera of the flexible endoscope, and an end cap adapted to open to allow passage therethrough of the video camera of the flexible endoscope and to close upon removal of the flexible endoscope therefrom.

19. The medical-treatment electrode assembly of claim 14, wherein the flexible tube is chosen from the group consisting of a transparent tube, a tube having a solid transparent window disposed between the two medical-treatment flexible electrodes, and a tube having a tube cutout disposed between the two medical-treatment flexible electrodes.

20. The medical-treatment electrode assembly of claim 19, also including a transparent flexible substrate bonded to the outer surface of the flexible tube, wherein the two medical-treatment flexible electrodes are bonded to the substrate.

21. A medical-treatment electrode assembly comprising a first medical-treatment electrode body which is insertable into a patient, which is operatively connectable to a medical radio-frequency (RF) generator, which has an outer surface contactable with patient tissue, which has a central lumen operatively connectable to a vacuum source, and which has an opening extending from the outer surface to the central lumen.

22. The medical-treatment electrode assembly of claim 21, also including a second medical-treatment electrode body insertable into the patient, operatively connectable to the medical radio-frequency (RF) generator, spaced apart from the first medical-treatment electrode body, having an outer surface contactable with the patient tissue, having a central lumen operatively connectable to the vacuum source, and having an opening extending from the outer surface of the second medical-treatment electrode body to the central lumen of the second medical-treatment electrode body.

23. A medical-treatment electrode assembly comprising:
a) a flexible tube insertable into a patient and including a sidewall having an opening;
b) a medical-treatment electrode supported by the flexible tube, contactable with patient tissue which is outside the flexible tube, and operatively connectable to a medical radio-frequency (RF) generator, wherein the flexible tube is operatively connectable to a vacuum source to draw patient tissue into the opening; and
c) two rollers disposed inside the flexible tube and adapted to rollingly engage patient tissue, drawn into the opening by the vacuum source, to draw more patient tissue into the flexible tube through the opening to tighten patient tissue outside the flexible tube against the medical-treatment electrode.

24. The medical-treatment electrode assembly of claim 23, wherein the opening is located closer to the two rollers than to the medical-treatment electrode.

25. The medical-treatment electrode assembly of claim 23, wherein the two rollers are translatable toward and away from each other.

26. A medical-treatment electrode assembly comprising:
a) a flexible tube insertable into a patient and including a sidewall having an opening;
b) two medical-treatment electrodes supported by the flexible tube, contactable with patient tissue which is outside the flexible tube, and operatively connectable to a medical radio-frequency (RF) generator, wherein the flexible tube is operatively connectable to a vacuum source to draw patient tissue into the opening, and wherein the two medical-treatment electrodes are spaced apart; and
c) two rollers disposed inside the flexible tube and adapted to rollingly engage patient tissue, drawn into the opening by the vacuum source, to draw more patient tissue into the flexible tube through the opening to tighten patient tissue outside the flexible tube against the two medical-treatment electrodes.

27. The medical-treatment electrode assembly of claim 26, wherein the two rollers are adapted to rollingly engage esophageal patient tissue, and wherein the two medical-treatment electrodes are contactable with esophageal patient tissue which is outside the flexible tube.

28. The medical-treatment electrode assembly of claim 27, wherein the opening is located closer to the two rollers than to the two electrodes.

29. The medical-treatment electrode assembly of claim 28, wherein the two rollers are translatable toward and away from each other.

30. A medical-treatment electrode assembly comprising:
a) a tube insertable into a patient and including a sidewall having an opening;
b) a medical-treatment electrode supported by the tube, contactable with patient tissue which is outside the tube, and operatively connectable to a medical radio-frequency (RF) generator; and
c) means for moving patient tissue into the opening to tighten patient tissue outside the tube against the medical-treatment electrode.

31. The medical-treatment electrode assembly of claim 30, wherein the means includes a vacuum source and does not include any roller.

32. The medical-treatment electrode assembly of claim 30, wherein the means includes two rollers and does not include a vacuum source.

33. The medical-treatment electrode assembly of claim 30, wherein the means includes a vacuum source and includes two rollers.

34. A method for medical treatment comprising the steps of:
a) inserting a tube into a hollow body organ of a patient, wherein the tube supports a medical-treatment electrode and has a sidewall opening;
b) then moving patient tissue into the sidewall opening to tighten patient tissue outside the tube into substantially full contact with the medical-treatment electrode; and
c) then activating the medical-treatment electrode.

35. A method for medical treatment comprising the steps of:
a) inserting a tube into an abdominal or thoracic cavity of a patient, wherein the tube supports a medical-treatment electrode and has a sidewall opening;
b) then moving patient tissue into the sidewall opening to tighten patient tissue outside the tube into substantially full contact with the medical-treatment electrode; and
c) then activating the medical-treatment electrode.
